(19) United States
(12) Patent Application Publication
(10) Pub. No.: US 2006/0154323 A1
(43) Pub. Date: Jul. 13, 2006

(54) Method for obtaining dynamic and structural data pertaining to proteins and protein/ligand complexes

(75) Inventors: Jonathan Miles Brown, Silver Spring, MD (US); Steven W. Homans, Leeds (GB); Minn-Chang Cheng, Clarksville, MD (US); Michael Chaykovsky, Columbia, MD (US); Jenny Hong Murray, Gaitersburg, MD (US)

Correspondence Address: ROTHWELL, FIGG, ERNST & MANBECK, P.C., 1425 K STREET, N.W., SUITE 800, WASHINGTON, DC, 20005, US

(73) Assignee: ProSpect Pharma, Columbia, MD (US)

(21) Appl. No.: 10/517,380

(22) PCT Filed: Jun. 10, 2003
(86) PCT No.: PCT/US03/18254

§ 371 Date: Jul. 7, 2005

Related U.S. Application Data
(60) Provisional application No. 60/386,739, filed on Jun. 10, 2002.

(51) Int. Cl. C12Q 1/37; C12Q 1/04; G01N 33/00; C07C 229/02

(52) U.S. Cl. 435/23; 435/34; 562/557; 436/86

(57) ABSTRACT

This invention provides an NMR method for obtaining both entropic and enthalpic data on proteins and protein/ligand complexes which can be used to obtain accurate structural and dynamic data of proteins and protein complexes having a wide range of molecular weights. An embodiment of the invention provides proteins which contain at least one bond vector whose dynamics are to be measured and which is surrounded by NMR inactive nuclei, and amino acids for synthesis of the proteins via chemical means or biological expression. The NMR methods using specifically labeled proteins for analysis result in maximization of the sensitivity and resolution of the NMR experiments, and minimization of the loss of signal due to diffusion.

### RELATED CASES

This application is based on and claims priority to U.S. provisional patent application No. 60/386,739, filed on Jun. 10, 2002, the entire contents of which are incorporated herein by reference.

### BACKGROUND OF THE INVENTION

1. Technical Field

This application relates to the field of drug design, and in particular to methods for obtaining dynamic and entropic data from specific locations of proteins and protein/ligand complexes over a wide range of molecular weights.

2. Description of the Background Art

The affinity of two molecules for each other is governed by an equation called the Gibbs'Free Energy Equation:
ΔG=ΔH−TΔS
where G is the Gibbs' free energy, H is the enthalpic (structural) component and S is the entropic (dynamic) component. If the change in Gibbs' free energy is negative, then the molecules will spontaneously bind. Conversely, if the Gibbs' free energy is positive on binding the molecules will immediately dissociate.

The Gibbs' free energy has two component parts. Either the structural or dynamic component can be dominant in the binding free energy of two molecules. In other words, a good structural fit between two molecules can be more than offset by an accompanying entropic penalty or, by contrast, the effect of a poor fit can be significantly enhanced by a favorable change in entropy on binding. For example, FIG. 1 shows the relative binding affinities of a panel of ligands to mouse urinary protein (MUP) at 298 K and the relative contributions of the enthalpic and entropic components of the Gibbs' free energy. Currently scientists in a number of fields are interested in obtaining a measure of the elusive entropic component of binding free energy, and have attempted to do so using a variety of biophysical methods, including isothermal titration calorimetry and molecular dynamics simulations. See, e.g., Schoen, Biochemistry 28:5019-5024, 1989; Chervenak and Toone, J. Am. Chem. Soc. 116:10533-10539, 1994; Dam et al., J. Biol. Chem. 273:32812-32817, 1998; Bundle et al., 120:5317-5318, 1998; Williams and Bardsley, Perspect. Drug Discov. Design 17:43-59, 1999; Shafer et al., 113:7809-7817, 2000; Caldesone and Williams, J. Am. Chem. Soc. 123:6262-6267, 2001; Harris et al., J. Am. Chem. Soc. 123:12658-12663, 2001; Clark et al., J. Am. Chem. Soc. 123:12238-12247, 2001; Schafer et al., 43:45-56, 2001.

These phenomena have a significant impact with regard to drug design. A drug is no more than a chemical entity which binds to a part of the human or pathogen biomolecular machinery, thereby impeding or imitating a biological function. Design of new drug molecules is exceptionally difficult since the molecule, to be effective, must bind tightly to the desired specific molecular target yet not bind to any of the vast number of other molecules of the body. Therefore, ignorance of the entropy change upon binding is an enormous handicap to discovering molecules which specifically and tightly bind to the target of interest.

The pharmaceutical industry has made a major attempt to improve the efficiency of drug design over the historically used methods of isolating and concentrating plant extracts and the like which were found to have desirable therapeutic effects, and attempting to improve the active ingredients through trial and error chemical modifications. Needless to say, these former processes were very slow and inefficient. The new methods include genomics (identifying all the component molecular parts of the human body), proteomics (identifying those parts of the biomolecular machinery involved in a given disease condition), combinatorial chemistry (synthesis of huge numbers of compounds which can be tested rapidly for desired characteristics using high throughput screening), and rational drug design (designing drugs on the basis of high resolution structural data of a molecular target of interest).

X-ray crystallography is widely used to obtain an estimate of the structure of proteins and can provide the complete tertiary structure (global fold) of the backbone of a crystallized protein. This method, however, has several disadvantages. For example, only proteins which can be crystallized may be studied using X-ray crystallography. Some proteins are very difficult or impossible to crystallize. Moreover, crystallization can be very time consuming and expensive. Another major disadvantage of this method is that the structural information obtained may be pertinent only to the crystalline structure of the protein and not to the structure of the protein in solution. The bond angles present in a crystal structure may not be the same as those of the protein when it is in an active conformation and therefore may not provide information relevant to the biological or physiological system of interest. Nor can this method provide any information whatsoever concerning the entropic component of protein folding or binding of a ligand.

High throughput screening of drug candidate compounds for binding of the target molecule by definition detects those compounds that have a favorable Gibbs' free energy change on binding to the target. But screening can not provide information concerning the enthalpic or entropic components of the Gibbs' free energy change. Conversely, X-ray crystallographic structural data can provide enthalpic data pertaining to the crystallized protein, but no dynamic data because the material must be crystallized (rigid) for the technique to work. The pharmaceutical industry therefore is forced to rely on multiple research projects, none of which is capable of supplying complete data needed for successful drug design. Even more importantly, the techniques available for use in rational drug design do not provide any information on the contribution of individual functional groups of protein or of ligand to the Gibbs' free energy change of binding of a ligand to a protein. In effect, drug designers presently must proceed without any information on many of the components of the biomolecular system that impact on the binding of the drugs under study. Therefore, a technique that can reliably and rapidly provide both complete enthalpic and entropic data on proteins would be highly useful.

Protein structure determination by high resolution multinuclear NMR also has become well known. In NMR spectroscopy, magnetization of certain atomic nuclei (usually protons) in a powerful magnetic field is detected by the absorption of radio waves. NMR has become a major tool in the study and analysis of small (<1 kD) molecules. For analysis of larger molecules such as proteins, it is essential in most applications to replace the natural abundance atoms of carbon and nitrogen (¹²C and ¹⁴N) universally with the NMR active stable isotopes ¹³C and ¹⁵N so that assignment of each of the detected NMR signals can be made reliably. See Ikura et al., Abstr. Pap. Am. Chem. Soc. 199:97-POLY (1990); Ikura et al., Abstr. Pap. Am. Chem. Soc. 199:107-INOR (1990); Bax, Curr. Opin. Struct. Biol. 4:738-744 (1994). Using isotopic labeling of this type, NMR has been used to determine the structure of several proteins. See Ikura et al., Biochemistry 30:9216-9228 (1991); Clore and Gronenborn, Nature Struct. Biol. 4:849-853 (1997). Moreover, because NMR can be carried out on a protein in solution, in contrast to X-Ray crystallography, which requires crystalline material, NMR studies can measure dynamic parameters. See Kay et al., Biochemistry 28:8972-8979 (1989).

In principle therefore, NMR can provide both enthalpic and entropic data on a drug target such as a protein. The actual use of NMR for these purposes, however, has been limited to the study of only relatively small molecules. Because the magnetization of the nuclei (¹H, ¹³C, ¹⁵N) in a protein tends to diffuse more easily with increasing molecular weight, the signal-to-noise ratio decreases with the size of the molecule being studied, rendering the data more difficult to interpret as the protein size increases. Thus, structure determinations of proteins have been restricted to sizes of about 35 kD or less and dynamics studies have been restricted to molecules of about 20 kD or less. In practice, this means that NMR has made only a modest impact to date on the drug design process. In essence this is because the very isotopes needed to assign the protein NMR signals in the first place, such as ¹³C, allow the magnetization to diffuse. In addition, the signals being assigned are split into multiplets by neighboring isotopes and this splitting further degrades the signal with respect to noise.

Attempts to ameliorate this degradation, for example using substitution with additional isotopes such as deuterium, can add complications due to the properties of their spin-states (which equals 1 in the case of deuterium). Thus measurement of deuterium relaxation, while attractive in view of theoretical simplicity, results in additional signal losses in NMR studies since only 66% of the signal energy can be transmitted to and from the deuterium nucleus, resulting in 43% efficiency. Muhandiram et al., J. Am. Chem. Soc. 117:11536-11544 (1995).

The difficulties encountered in these NMR structural determinations are due largely to using proteins for study which are universally isotopically enriched. Such proteins are used because the labeling systems that are currently commercially available yield only random, universal enrichment. Such universal labeling yields split signals in the NMR spectrum, each of which need to be assigned before a structure determination can be commenced. Splitting of signals results in both more and weaker signals. This phenomenon causes overlap of signals and a far inferior signal-to-noise ratio, both of which make the assignment process more difficult and both of which are greatly increased with protein size. Therefore, in practice these methods can provide fairly accurate structures only of small proteins.

Recently methods have been described which attempt to overcome these problems and dramatically increase the resolution and sensitivity of NMR spectra in structural studies of proteins. These methods utilize specific isotopic enrichment of the protein in the backbone only, which greatly facilitates both the detection and assignment of the NMR signals and the calculation of the structure of the protein. (Coughlin et al., J. Am. Chem. Soc. 121, 11871-11874, 1999; Giesen et al., J. Biomol. NMR 19, 255-260, 2001; Giesen et al., J. Biol. NMR., pp. 1-9, 2002.

Several studies have been undertaken to develop NMR methods for study of protein dynamics. These studies on entropic contribution to binding have focused on side-chain methyl groups in protein. These studies have used either ¹³C or ²H relaxation measures in the ¹³CH²D or ¹³CHD₂ isotopomers respectively. Lee et al., Nature Str. Biol. 7:72-77, 2000; Muhandiram et al., J. Am. Chem. Soc. 117:11536-11544, 1955; Yang et al., J. Mol. Biol. 276:939-954, 1998; Mittermaier et al., J. Biomol. NMR 13:181-185, 1999; Lee et al., J. Am. Chem. Soc. 121:2891-2902, 1999; Ishima et al., J. Am. Chem. Soc. 121:11589-11590, 1999; Ishima et al., J. Am. Chem. Soc. 123:6164-6171, 2001; Skrynnikov et al., J. Am. Chem. Soc. 123:4556-4566, 2001.

However, as with the structural studies, there are sensitivity problems with all the approaches tried so far which have limited the measurement of dynamics and entropy to very small proteins. Although compelling reasons exist to choose ²H relaxation measurements where possible, in practice ¹³C relaxation measurements are considerably more sensitive. Muhandiram et al., J. Am. Chem. Soc. 117:11536-11544, 1995; Lee et al., J. Am. Chem. Soc. 121:2891-2902, 1999. Indeed, in studies using the mouse major urinary protein (MUP) as a model system for thermodynamics of ligand-protein interactions, considerable difficulty was experienced in measuring accurate ²H relaxation rates for valine methyl groups in methyl ¹³C, 50% ²H-enriched protein due to the combined effects of resonance overlap and relatively poor sensitivity, despite the only modest size of this protein (˜19 kD). The low sensitivity derived from the fact that only the ¹³CH₂D isotopomer was detected in ²H relaxation measurements, whereas the sample contained all possible ²H isotopomers, even with optimized labeling schemes. See Ishima et al., J. Biomol. NMR 21:167-171, 2001. Consequently, the effective protein concentration in the sample was reduced considerably.

Therefore, because sensitivity is especially important in larger proteins, ¹³C relaxation measurements are the only viable approach for such molecular systems. ¹³C relaxation studies on side-chain methyl groups in proteins typically have involved ¹³CHD₂ isotopomers, where the ¹³C relaxation mechanism is particularly straightforward in the presence of a single proton. While such isotopomers can be obtained in proteins overexpressed in bacteria by use of conventional ¹³C-enriched and fractionally deuterated media, again all possible ²H isotopomers are obtained in these systems. Thus the desired isotopomer (¹³CHD₂) is diluted by the undesirable ones (¹³CH₂D, ¹³CH₃, ¹³CD₃). This results in a loss of both resolution and sensitivity, which becomes particularly severe for even modest-size proteins, for example proteins of 20 KD or greater.

Additional problems can result from attempting to measure ¹³C relaxation rates in partially deuterated proteins with multiple isotopomers. Thus, while ¹³C relaxation measurements on the MUP system cited above resulted in much higher sensitivity, resonance overlap was very severe, due in part to the presence of contaminating resonances from ¹³CH₃ isotopomers in refocused INEPT studies designed to selectively detect resonances from ¹³CHD₂ isotopomers. The ¹³CH₃ isotopomers resonate at a different chemical shift from the ¹³CHD₂ isotopomers due to the deuterium isotope effect, and are incompletely suppressed in refocused INEPT studies as a consequence of the different relaxation rates in proteins of the 3/2 and 1/2 spin manifolds of the ¹³C spin in CH₃ groups.

Thus, to overcome resonance overlap difficulties of this type and the associated lack of sensitivity, a new labeling strategy heretofore unavailable is required to measure the dynamics and associated entropy values for apo-proteins and proteins complexed with ligands such as drug candidates. Therefore, there is a need for a technology platform that can provide both enthalpic and entropic data on a drug target such as a protein and particularly such proteins bound to a ligand such as a drug or drug candidate. There also is a need to provide methods for NMR spectroscopy to achieve significant enhancement of the size range over which dynamic data can be obtained for proteins and their complexes with drug candidates. A system that can do so on a functional group by functional group basis as an aid to drug design would be particularly useful.

### SUMMARY OF THE INVENTION

Accordingly, embodiments of the invention provide methods and materials which can be used to obtain dynamic data on proteins and protein/ligand complexes over a wide range of molecular weights (such as for example 10 kD to 150 kD or more), including membrane proteins and multi-protein complexes. In particular, embodiments of the invention provide methods for significantly enhancing the sensitivity and resolution of measurements of dynamics of a protein and protein/ligand complexes using NMR spectroscopy which allows information to be obtained on large proteins and protein systems and which allows the contribution of single functional groups with respect to binding to be examined.

One aspect of the invention provides proteins which contain at least one isotopically labeled bond vector the dynamics of which are to be measured and which is surrounded by NMR inactive bond vectors. In this way the sensitivity and resolution of the NMR experiments is maximized, while the loss of signal due to diffusion is minimized.

Another aspect of the invention provides methods for preparing and analyzing proteins which are composed essentially completely of ¹²C, ¹⁴N and ²H nuclei, save for isolated ¹³C—H vectors at positions in the side chains of amino acids in the protein which it is desired to study by NMR.

A further aspect of the invention provides methods for preparing and analyzing proteins which are composed essentially completely of ¹²C, ¹⁴N and ²H nuclei, save for isolated ¹⁵N—H vectors at positions in the side chains of amino acids which it is desired to study by NMR.

A particularly preferred aspect of the invention provides a protein which is composed essentially completely of ¹²C, ¹⁴N and ²H nuclei, save for isolated ¹³C—H vectors at positions in the side chains of amino acids in the protein which it is desired to study by NMR. In another particularly preferred aspect of the invention, a protein is provided which is composed essentially completely of ¹²C, ¹⁴N and ²H nuclei, save for isolated ¹⁵N—H vectors at positions in the side chains of amino acids in the protein which it is desired to study by NMR.

A further aspect of the invention provides methods for the chemical synthesis of amino acids which are composed completely of ¹²C, ¹⁴N and ²H nuclei, save for isolated ¹³C—H vectors at positions in the side chains of the amino acids which it is desired to study by NMR.

A further aspect of the invention provides methods for the chemical synthesis of amino acids which are composed completely of ¹²C, ¹⁴N and ²H nuclei, save for isolated ¹⁵N—H vectors at positions in the side chains of the amino acids which it is desired to study by NMR.

Yet another aspect of the invention provides methods for the culture of cells in media containing specifically labeled amino acids, which provide for the prevention of isotopic scrambling.

Embodiments of the invention provide an amino acid wherein at least one bond vector in the side chain of the amino acid consists of two NMR-active nuclei bonded together and wherein essentially all other nuclei are NMR inactive. Embodiments of the invention further provide an amino acid wherein at least one bond vector in the side chain of the amino acid consists of two NMR active nuclei bonded together and wherein essentially all other bond vectors are NMR inactive.

Embodiments of the invention further provide an amino acid as described above wherein the two NMR-active nuclei are ¹³C and ¹H, wherein the remainder of the carbon atoms in the amino acid are essentially ¹²C, wherein the nitrogen atoms in said amino acid are essentially ¹⁴N and wherein the remainder of the hydrogen atoms in the amino acid are essentially ²H. Embodiments of the invention also provide an amino acid as described above wherein the two NMR-active nuclei are ¹³C and ¹H, wherein the remainder of the carbon atoms in the amino acid are essentially ¹²C, wherein the nitrogen atoms in said amino acid are essentially ¹⁴N, wherein the carbon atoms in the amino acid are essentially ¹²C and wherein the remainder of the hydrogen atoms in said amino acid are natural abundance. Embodiments of the invention further provide an amino acid as described above wherein the two NMR-active nuclei are ¹⁵N and ¹H, wherein the remainder of the nitrogen atoms in the amino acid are essentially ¹⁴N, wherein the carbon atoms in the amino acid are essentially ¹²C and wherein the remainder of the hydrogen atoms in the amino acid are essentially ²H. Embodiments of the invention also provide an amino acid as described above wherein the two NMR-active nuclei and ¹⁵N and ¹H, wherein the remainder of the nitrogen atoms in said amino acid are essentially ¹⁴N, wherein the remainder of the carbon atoms in the amino acid are essentially ¹²C and wherein the remainder of the hydrogen atoms in the amino acid are natural abundance.

Embodiments of the invention also provide a culture medium suitable for growth of protein-producing cells, such as bacteria, yeast, mammal or insect cells that comprise amino acids as described above and proteins that comprise at least one amino acid as described above.

Further, embodiments of the invention provide a method of analyzing the dynamics of a bond vector of a protein comprising producing the protein in a form which comprises an amino acid as described above and subjecting the protein to NMR spectroscopy. Another embodiment of the invention provides a method of determining the entropic contribution of a bond vector of a protein bound to a ligand comprising producing the protein in a form which comprises an amino acid as described above and subjecting the protein to NMR spectroscopy in the presence and the absence of the ligand.

Embodiments of the invention further provide a method of preparing an isotopically substituted protein comprising culturing cells that express the protein in a medium containing at least one amino acid as described above and recovering the protein from the culture medium or from the cells.

### BRIEF DESCRIPTION OF THE DRAWINGS

FIG. 1 provides the relative binding affinities and Gibbs' free energy components of a panel of ligands to mouse urinary protein at 298 K.

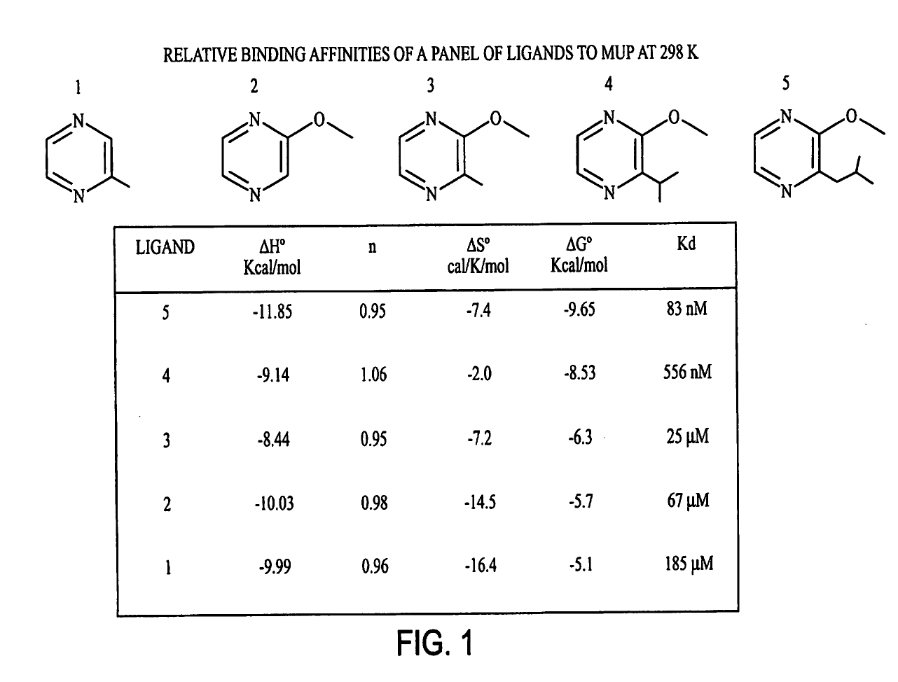

FIG. 2 provides an example for the preparation of a precursor used in the synthesis of specifically labeled valine.

FIG. 3 provides an example for the preparation of specifically labeled amino acid.

FIG. 4 is an example of preparation of specifically labeled amino acids.

FIG. 5 is an example of preparation of intermediates for the synthesis of specifically labeled amino acids.

FIG. 6 provides a synthetic scheme for chemical synthesis of L-valine-α-D-¹²CD(¹³CHD₂)₂.

FIG. 7 is an SDS-PAGE gel (lane 1: molecular weight standards; lane 2: non-induced MUP; lane 3: induced MUP; lane 4: insoluble cell break pellet; lane 5: Ni-NTA column flow-through; lane 6: Ni-NTA column elution; lane 7: anion exchange fraction 1; lane 8: anion exchange fraction 2.

FIG. 8 shows NMR results for mouse urinary protein expressed with L-valine-α-D-¹²CD(¹³CHD₂)₂.

FIG. 9 shows a region of the ¹³C—¹H HSQC spectrum of methyl-¹³C, 50%-¹²H enriched MUP showing valine methyl correlations (9A); an equivalent spectrum of MUP selectively enriched with (¹³C^{γ1γ2}HD₂)₂¹²C^{α,β}D-L-valine (9B); and an overlay of regions of the ¹³C—¹H HSQC spectra of (¹³C^{γ1γ2}HD₂)₂¹²C^{α,β}D-L-valine enriched MUP in complex with 2-methoxy-3-isopropylpyrazine (black correlations) and 2-methoxy-3-isobutylpyrazine (gray correlations) (9C).

FIG. 10 provides typical relaxations curves for Val-12C^{γ1} obtained from (¹³C^{γ1γ2}HD₂)₂¹²C^{α,β}D-L-valine enriched MUP; solid line: free protein; dashed line: 2-methoxy-3-isopropylpyrazine complex; dash-dotted line: 2-methoxy-3-isobutylpyrazine complex. For clarity, only the data points corresponding to the free protein are shown. Error bars are smaller than the symbols used for these data points.

### DETAILED DESCRIPTION OF THE PREFERRED EMBODIMENTS

Specific labeling of a protein in the backbone has been shown to be very effective in providing high resolution and sensitive data to expedite the assignment of the backbone signals of a protein (Coughlin et al., J. Am. Chem. Soc. 121, 11871-11874, 1999; Giesen et al., J. Biomol. NMR 19, 255-260, 2001; and also the determination of the Global Fold (Giesen et al., J. Biol. NMR, pp. 1-9, 2002). In a like manner, embodiments of the present invention provide for specific labeling in the side chains of amino acids in a protein or protein/ligand complex, thereby increasing the sensitivity and resolution of NMR studies to determine the dynamics of relevant amino acid sidechains. Embodiments of the present invention further provides methods to produce amino acids that contain a pure ¹³CH₂ isotopomer, e.g. (¹³C^{γ1γ2}HD₂)₂¹²C^{α,β}D-L-valine, the most sensitive isotopomer possible. Moreover, embodiments of the present invention provide methods for the isotopomer to be contained in an NMR “invisible” environment, thereby maximizing resolution and increasing sensitivity still further. Protein is prepared by including the desired amino acid in an appropriate bacterial, yeast, insect or mammalian growth medium for growth of cells that express or overexpress the desired protein. The resulting protein contains isotopically enriched atoms only in the desired species of amino acid, e.g., valine or lysine, thereby maximizing the resolution and sensitivity of the NMR studies.

The invention provides a means for rapidly determining by NMR the dynamics of the sidechain of a protein, or protein/drug complex, of a size considerably larger than heretofor. The term “dynamics” refers to the entropic component of particular atoms or bond vectors in a protein with respect to three dimensional structure and information of the protein, with or without binding of a ligand. The invention allows this information to be obtained by increasing the resolution of signals of interest from one or more pairs of atoms in one or more amino acids in the protein while simultaneously reducing the tendency of the magnetization of these atoms in an NMR study to diffuse. This is accomplished by specifically labeling one or more of the amino acids in the protein in the side chain with one or more pairs of NMR active nuclei (such as ¹H, ¹³C or ¹⁵N, for example) that are covalently bonded together. All other atoms of both the side chain and the backbone of the amino acid preferably are selected to minimize sensitivity losses and to increase resolution. Preferably, the bonded pairs of NMR active atoms in the labeled amino acid are ¹H and ¹³C or ¹H and ¹⁵N, such as those contained in —¹³CHD₂-, —¹³CHD-, —¹³CH— and —¹⁵NH— groups and all other nuclei in the protein are essentially ¹²C, ¹⁴N and D (²H, deuterium). The methods and compositions of embodiments of this invention are isotopically substituted or enriched so that a single bond vector is composed of two NMR active nuclei when all other bond vectors are NMR inactive. Preferably, all other atoms are NMR inactive.

The term “active,” when referring to NMR-active nuclei is used according to the common usage in the art of NMR studies. Natural abundance refers to the isotopes of an atom that occur in nature. One of skill in the art will recognize that atoms do not exist in a single isotope in nature, and therefore that an atom such as carbon, for example will exist as ¹²C for the most part, but also will exist to a certain degree as ¹³C, naturally. Therefore a carbon-containing molecule that is unlabeled nevertheless will contain a small amount of other isotopes as well. Thus, a carbon position in a molecule that is essentially ¹²C contains ¹²C in the same or essentially the same ratio (abundance) as occurs in nature. Other atoms such as nitrogen and hydrogen also occur naturally as different isotopes and therefore the terms “natural abundance” and “essentially” may be understood an analogous fashion with respect to any atom. One of skill in the art also will recognize that an isotopically substituted atom also is not 100% of the stated isotope but rather is enriched in the stated isotope. The term enriched refers to an isotope that is greater than natural abundance, up to about 5-100%, preferably about 5-20% or about 10-20% or most preferably about 10%.

Although the methods of this invention are suitable for the determination of dynamic information of any peptidic molecule of three or more amino acids in length, and therefore encompasses both proteins and peptides, the description, for simplicity, will refer only to proteins. It is understood that the term “protein,” as used in this application, refers to any peptidic molecule of three or greater amino acids, or, for example, peptides and proteins of about 5 kD or greater molecular weight. The compositions and methods of the present invention therefore advantageously may be employed in connection with proteins having molecular masses of about 5 kD or more, or proteins of about 50 amino acid residues or more. The methods are particularly useful for proteins of 20-30 kD or larger, which have been difficult to study using prior art methods, and even more particularly proteins of 50 or 55 kD or more or 75 kD, or proteins of 100 kD or longer. Therefore any protein of 50, 60, 70, 80, 90, 100, 110, 120, 130, 140, 150 kD or more, or complexes of such proteins, are suitable for structural and dynamic information determinations according to embodiments of this invention. The methods may be used to study membrane proteins as well. Of course, smaller proteins and peptides may be studied using the inventive methods, including oligopeptides and any peptide of three or greater amino acids. Proteins containing the specifically labeled amino acids may be chemically synthesized from scratch or beginning with natural amino acids, or expressed by cells in culture, for example by bacterial, yeast, mammalian or insect cells.

The amino acids of the protein are labeled at specific positions with any combination of the NMR-relevant isotopes ²H, ¹³C and/or ¹⁵N such that only those atoms required to be visible in the spectrum are detected. Those skilled in the art will recognize that a key step required in the elucidation of protein dynamics by NMR is the measurement of the rate of decay of magnetization from a bond vector, such as for example a C—H or a N—H bond vector. The vector to be measured is labeled with ¹³C or ¹⁵N, to form a ¹³C—H or ¹⁵N—H bond vector in the amino acid or acids which are desired to be analyzed. Any bond vector can be specifically labeled with an appropriate isotope, such as the NMR-active isotopes ¹H, ¹³C, ¹⁵N, ¹⁷O or any other necessary isotope, while the remainder of the bond vectors are NMR inactive and consist of ²H(D), ¹²C, ¹⁴N, ¹⁶O, etc. This is in contrast to earlier methods where amino acids where labeled either universally by isotope type, e.g. with commercially available ¹⁵N₂-lysine (Cambridge Isotope Laboratories) or were partially but randomly labeled during protein synthesis. (Rosen, M. K., Gardner, K. H., Willis, R. C., Parris, W. E., Pawson, T., and Kay, L. E. (1996) J. Mol. Biol. 263, 627-636. Gardner, K. H., Rosen, M. K., and Kay, L. E. (1997) Biochemistry 36, 1389-1401. Gardner, K. H., and Kay, L. E. (1997) J. Am. Chem. Soc. 119, 7599-7600).

The rate of decay of magnetization is an inverse function of the dynamic energy of the bond vector. The bond vector to be labeled for analysis by NMR may be any bond vector of interest in the protein. However, for preference the bond vector of choice often will be near, and preferably at, the terminus of an amino acid side chain for maximum sensitivity when ligand-binding areas of a protein are to be studied.

It will further be appreciated by those skilled in the art that many of the side chains of amino acids are composed of (CH₃)—, and —(CH₂)— groups. Therefore, for maximum sensitivity of a particular C—H bond vector in such a group, the other protons covalently attached at that group are preferably replaced with deuterium atoms such that the C—H bond vector of interest is isolated and its retention of magnetization enhanced. Therefore, it is particularly preferred that the C—H bond vectors desired to be studied are labeled as follows: —¹³CHD₂-, —¹³CHD- and —¹³CH—. Preferably, all other carbon, nitrogen, and hydrogen atoms of the amino acids of the protein are NMR inactive (e.g. ¹²C, ¹⁴N, and D (deuterium)). In another particularly preferred embodiment of the invention, the isolated bond vectors for study are ¹⁵NH or ¹⁷OH, with all other atoms of the amino acids of the protein NMR inactive, analogous to the strategy described above.

Amino acids have been chemically synthesized in unlabeled forms by various means and some have been synthesized in specifically isotopically labeled forms (for reviews see Martin, Isotopes Environ. Health Stud, 32, 15, 1996, Schmidt, Isotopes Environ. Health Stud, 31, 1995, 161). Thus Ragnarsson et al. J. Chem. Soc. Perkin Trans 1: 2503, 1994) synthesized 1,2-¹³C₂, ¹⁵N Ala, Phe, Leu, Tyr, 1,2-¹³C₂, 3′,3′,3′-²H₃, ¹⁵N Ala and 1,2-¹³C₂, 3′,3′-²H₂, ¹⁵N Phe and 3′,3′,3′-²H₃ Ala. Ragnarsson (17) also synthesized 1,2-¹³C₂, 2-²H, ¹⁵N Ala, Leu and Phe and 1,2-¹³C₂, 2,2-²H₂, ¹⁵N Gly, these were partly used for conformational studies of a pentapeptide, Leu-enkephalin. Unkefer synthesized ¹⁵N labeled Ala, Val, Leu, Phe as well as 1-¹³C, ¹⁵N Val. More recently, methods for the preparation of backbone labeled amino acids have been developed and these have been used for the assignment of backbone signals as well as to determine the backbone Global Fold of a protein. In all these cases, stereo-selective addition of the appropriate amino acid sidechain was added to glycine derivatized in a chiral complex.

Amino acid precursors for selective protonation of certain ¹³C-labeled amino acids in proteins during cell culture have been described. These materials, ¹³C₄-3,3-²H₂-α-isobutyrate and ¹³C₅-3-²H-α-isoketovalerate, have been used to produce ¹³CH₃-methyl leucine, isoleucine and valine residues in a perdeuterated protein expressed in bacteria. Rosen et al., J. Mol. Biol. 263, 627-636, 1996; Gardner et al., Biochemistry 36, 1389-1401, 1997; Gardner et al., J. Am. Chem. Soc. 119, 7599-7600, 1997. Very recently, unlabeled α-isobutyrate and α-isoketovalerate containing terminal ¹³CH₃-groups also have been described for much the same purpose. Hajduk et al., J. Am. Chem. Soc. 122, 7898-7904, 2000. All these methods and materials have increased the scope of analysis of proteins by NMR, however these labeling methods and the NMR analytical procedures they allow do not provide the maximum possible sensitivity of the NMR analysis, nor are they applicable for every possible protein type. Moreover, not all the labeling patterns are applicable for every amino acid type. For instance, ¹³CH₃-methyl leucine, isoleucine and valine residues in a perdeuterated environment is of value only to studies involving those residue types or those close to them. Other residues, including other hydrophobic species such as phenylalanine of potential interest to binding studies, cannot be studied in this way.

Therefore, a particularly preferred aspect of the present invention is a method for the synthesis of all twenty amino acids specifically labeled in the side chain with (¹³CHD₂)- and/or —(¹³CHD)- and/or —(¹³CH)— and/or —(¹⁵NH)-moieties, all other parts of the amino acids of the protein being essentially in the form of the nuclei ¹²C, ¹⁴N and deuterium. Amino acids specifically labeled in this way may be synthesized by asymmetric synthesis from glycine such as those cited above using an appropriately isotopically labeled sidechain precursor. Precursors such as ¹³CHD₂-labeled methyl iodide are available commercially. Preferably therefore, the amino acids are synthesized from glycine using side chain precursors themselves prepared from specifically labeled precursors such as ¹³CHD₂-labeled methyl iodide.

As noted, many methods for the synthesis of amino acids from glycine have been described (Duthaler, Tetrahedron 50, 1539, 1994; Schöllkopf, Topics Curr. Chem., 109(65): 1983; Oppolzer, Tett. Letts., 30:6009, 1989; Helvetica Chimica Acta, 77:2363, 1994; Helvetica Chimica Acta 75:1965, 1992) and these may be used in accordance with the present invention. In a preferred aspect of the invention, however, glycine is first converted to a nickel II transition metal complex according to the methods of Belokon et al. (J. Chem. Soc. Perkin. Trans. 1: 1525-1529, 1992). The derivatized glycine then is alkylated by treatment with a base, such as sodium hydroxide, sodium methoxide or preferably, potassium t-butoxide, followed by addition of the appropriate sidechain precursor. The precursor is an alkyl chain containing a ¹³C—¹H and/or ¹⁵N—H label where required and ¹²C, ¹⁴N and ²H in all other positions, bonded to chemical leaving group such as bromide, iodide, 4-nitrobenzenesufonate, etc. at the appropriate position.

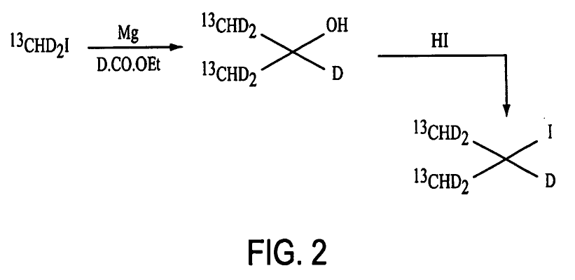

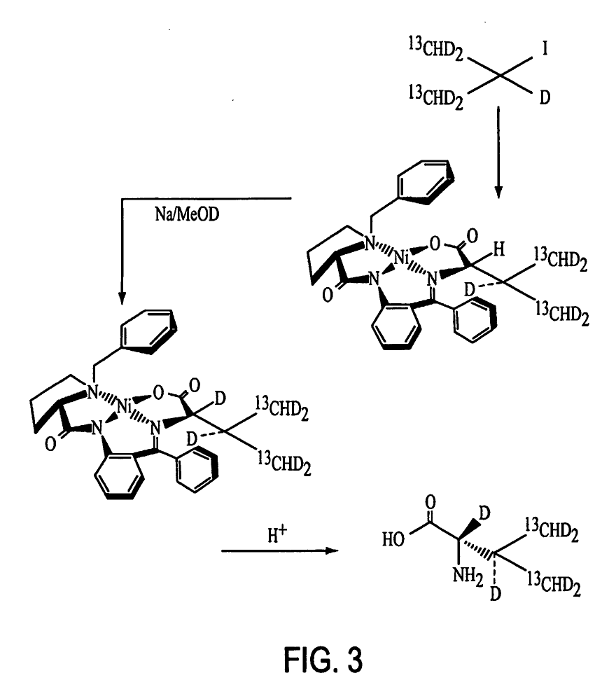

Precursors of this type can be readily synthesised from commercially available materials. Thus, (¹³CHD₂)₂—CD-iodide, the desired precursor for specifically labeled valine, can be prepared from ¹³CHD₂-labeled methyl iodide via a Grignard reaction with magnesium and deuterated ethyl formate, and halogenation of the resulting specifically labeled isopropyl alcohol. See FIG. 2. This specifically labeled sidechain then can be added to glycine derivatized as a chiral complex with the desired specifically labeled valine being obtained via acid hydrolysis. See FIG. 3.

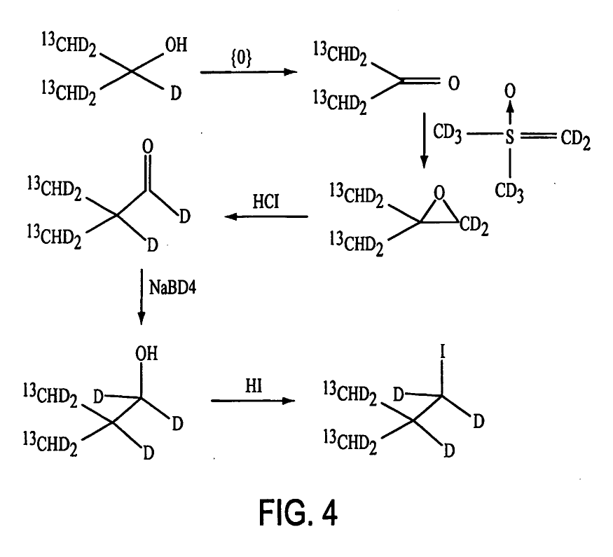

Alternatively, the specifically labeled isopropanol can be oxidized to the correspondingly labeled acetone and the carbon chain extended by treatment with a methylene donor such as dimethyloxosulfonium methylide to yield the required precursor for specifically labeled leucine. See FIG. 4. Addition of the iodide to the glycine complex as above yields specifically ¹³CHD₂-labeled leucine.

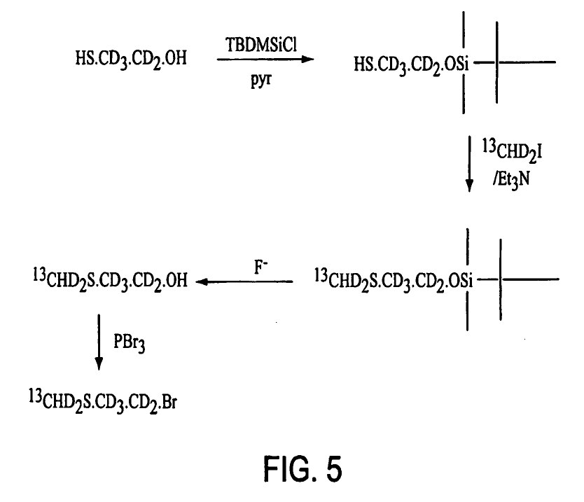

Alternatively, ¹³CHD₂-iodide can be reacted with protected beta-mercapto ethanol shown in FIG. 5. Reaction of the specifically ¹³CHD₂-labeled thio ether with the glycine complex and subsequent acid hydrolysis yields methionine. In this way, specifically labeled sidechains of all the alkyl amino acids can be constructed.

The following examples are provided to illustrate and not to limit the invention claimed herein.

### EXAMPLES

### Example 1

### Synthesis of L-(¹³C^{γ1γ2}HD₂)₂¹²C^{α,β}D-L-Valine

Magnesium turnings (6.08 g, 250.00 mmol, 2.50 equiv.) and anhydrous ether (100 mL) were added into a 3-neck 500 ml round bottom flask equipped with condenser, mechanical stirrer and heating mantle. The mixture was stirred and heated until under gentle reflux. ¹³CHD₂-I (Cambridge Isotope Labs, 28.99 g, 200.00 mmol, 2.00 equiv.) in anhydrous ether (50 mL) was added dropwise into the Mg/ether mixture over 30 minutes and refluxing was continued for another 2 hours with the heating mantle to form a Grignard reagent. The reaction was then cooled in an ice bath.

D-CO—OCH₃ (Cambridge Isotope Labs), 6.11 g, 100.00 mmol, 1.00 equiv.) in anhydrous ether (50 mL) was added slowly into the Grignard reagent over 15 minutes. The ice bath was removed and the reaction mixture was stirred for another 4 hours. The reaction mixture then was cooled again in an ice bath and saturated aqueous NH₄Cl solution (35 mL) was added slowly over 15 minutes to quench the Grignard reaction. The reaction mixture was filtered and the solid was rinsed with ether (2×100 mL). The combined ether solutions were dried (MgSO₄) and filtered. The filtrate was distilled slowly and carefully through a 10-inch column containing glass helices. When the temperature of the distillate reached 40° C., the remaining liquid was transferred into a 15 mL pear-shaped flask and distilled through a 1-inch Vigreaux column to give (¹³CHD₂)¹²CD-iodide (1.46 g, 21.76 mmol, 21.76% yield, F.W.=67.11; bp 78-82° C.).

BPB-Ni(II)-Gly red complex (7.22 g, 14.49 mmol, 1.00 equiv.), prepared essentially by the method of Belokon et al., was suspended in anhydrous CH₃CN (200 mL) at room temperature. (¹³CHD₂)¹²CD-iodide (2.83 g, 15.99 mmol, 1.10 equiv.) in anhydrous CH₃CN (10 mL) was added to the red reaction suspension, followed after 5 minutes by NaO^{t}Bu (1.54 g, 16.02 mmol, 1.11 equiv.). After 4 hours, thin layer chromatography (silica gel, acetone/CHCl₃=1/5) revealed the presence of a trace of unreacted starting material and a major spot with a higher R_{f} value. Therefore, glacial acetic acid (3.84 g, 3.66 mL, d=1.049, 63.95 mmol, 4.41 equiv.) was added to quench the reaction.

The reaction mixture was concentrated under reduced pressure and poured into a 2 L Erlenmeyer flask containing H₂O (1 L). The mixture was extracted with CH₂Cl₂ (3×200 mL) and the combined organic layers were washed with H₂O (2×200 mL) and then brine solution (200 mL). The organic phase was dried (MgSO₄) and evaporated to provide a red crude foamy glass. The crude product was subjected to further purification by flash column chromatography on silica gel using chloroform:acetone as eluant. The appropriate fractions were combined and evaporated to dryness. The residue was dissolved in 1:1 toluene:MeOD (Isotec, 100 ml), treated with sodium metal (200 mg) and the whole heated to reflux overnight. On cooling, the reaction mixture was treated with deutero-acetic acid (Cambridge Isotope Labs, 1 mL) and concentrated under reduced pressure. The mixture was extracted with CH₂Cl₂ (3×200 mL) and the combined organic layers were washed with H₂O (2×200 mL) and then brine solution (200 mL). The organic phase was dried (MgSO₄) and evaporated. The resulting red foamy glass was dissolved in methylene chloride (10 ml) and added dropwise to stirred hexane (2 L). The suspension was stirred overnight, filtered and the collected solid dried to provide (¹³C^{γ1γ2}HD₂)₂-¹²C^{α,β}D-L-valine-Ni(II)-BPB (4.32 g, 7.89 mmol, 54.45% yield).

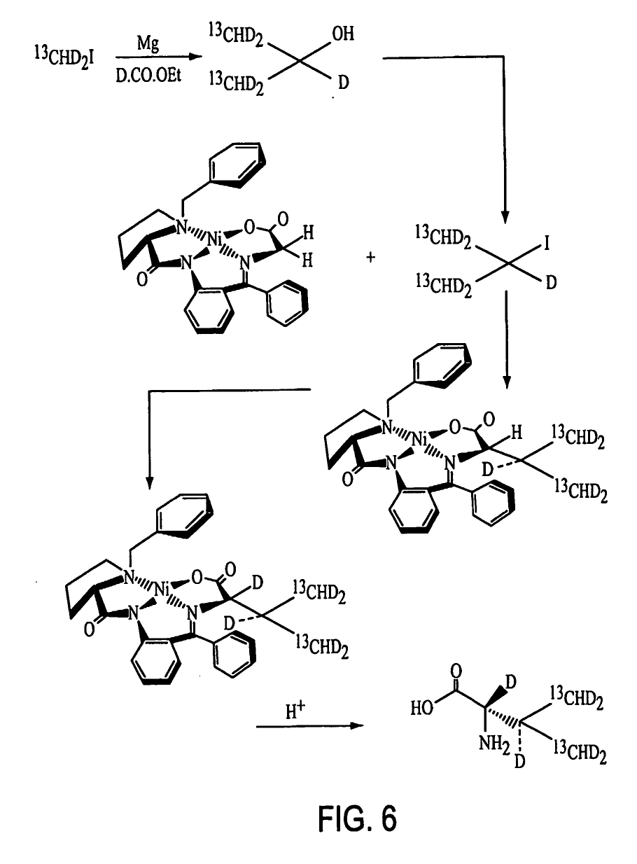

This complex (4.32 g, 7.89 mmol, 1.00 equiv.), CH₃OH (60 mL), and 2 M HCl (60 mL) were heated at reflux for 10 minutes. The pale green solution was evaporated to dryness on a rotary evaporator. H₂O (50 mL) was added to the dried solid. The mixture was cooled in an ice bath for several hours, followed by filtration. Thin layer chromatography (silica gel, BuOH/acetic acid/water=2/1/1) of the filtrate showed the presence of the title compound (¹³C^{γ1γ2}HD₂)₂-¹²C^{α,β}PD-L-valine. The filtrate was dried and the title compound was isolated by ion exchange chromatography and crystallized from aqueous ethanol (0.74 g, 5.91 mmol, F.W.=125.16, 75% yield from BPB-Ni(II)-glycine). See FIG. 6.

### Example 2

### Expression and Purification of Murine Urinary Protein (MUP) Containing L-valine-α-D-¹²CD(¹³CHD₂)₂

A 20 mL stock culture of M15 cells transformed with the vector pqe30 MUP was used to inoculate 1 L of medium containing 500 mg alanine, 400 mg arginine, 400 mg aspartic acid, 50 mg cysteine, 400 mg glutamine, 650 mg glutamic acid, 550 mg glycine, 100 mg histidine, 230 mg isoleucine, 230 mg leucine, 420 mg lysine HCl, 250 mg methionine, 130 mg phenylalanine, 100 mg proline, 2.1 g serine, 230 mg threonine, 170 mg tyrosine, 230 mg valine, 500 mg adenine, 650 mg guanosine, 200 mg thymine, 500 mg uracil, 200 mg cytosine, 1.5 g sodium acetate (anhydrous), 1.5 g succinic acid, 750 mg NH₄Cl, 850 mg NaOH, 10.5 g K₂HPO₄ (anhydrous), 2 mg CaCl₂ 2H₂O, 2 mg ZnSO₄ 7H₂O, 2 mg MnSO₄H₂O, 50 mg tryptophan, 50 mg thiamine, 50 mg niacin, 1 mg biotin, 20 g glucose, 4 mL 1 M MgSO₄, 1 mL 0.01 M FeCl₃, 15 mg ampicillin, and 50 mg kanamycin.

When cell density had reached an OD of 1.2, the cells were harvested by centrifugation, rinsed with PBS, recentrifuged and resuspended in a medium of the above proportions but in which L-valine-α-D-¹²CD(¹³CHD₂)₂ was substituted for the unlabeled valine. After 30 minutes, protein expression was induced by addition of IPTG to a final concentration of 0.1 mmol. After 6 hours, the cultured cells were centrifuged at 4000 rpm for 20 minutes in a Sorvall RC-3B centrifuge. The cell pellet was then stored at −20° C. overnight. The cells were thawed and resuspended in 30 ml of sonication buffer (50 mM sodium phosphate, 500 mM NaCl, pH=8.0). The cells were broken by passing them through a French Press four times at 20,000 psi. The broken cells were subjected to sedimentation at 15,000×g for 20 minutes in Oakridge tubes.

A 5 mL Ni-NTA immobilized metal affinity column was equilibrated in sonication buffer at 5 mL/min. The supernatant (cell lysate) was removed from the Oakridge tube without disturbing the pellet. Cleared lysate was loaded onto the column at 5 ml/min. The column flow-through was saved for later analysis. The material bound to the column was then washed with sonication buffer for 30 minutes until the absorbance of the effluent was less than 0.020. Bound protein was eluted from of the column with Elution Buffer (50 mM sodium phosphate, 500 mM NaCl, 500 mM imidizole, pH=8.0) using a single step. The peak fraction was collected manually. A sample of the elution was saved for analysis.

A 300 mL XK-50 column packed with Sephadex G-25 Fine size exclusion chromatography (SEC) resin was equilibrated with anion exchange Buffer A (20 mM Tris HCl, pH=7.5). The Ni-NTA eluate was loaded onto the SEC column at 15 ml/min. The protein peak was collected manually. The protein sample, now in anion exchange Buffer A, was stored at 4° C. during preparation of the next step. A 10 ml Resource Q anion exchange column was equilibrated in Buffer A. The partially purified protein was loaded onto the column at 10 mL/min. The sample was washed with Buffer A for three minutes. The sample was eluted with a linear gradient into Buffer B (20 mM Tris HCl, 1 M NaCl, pH=7.5) over seven minutes. The fractions were collected in 30 second intervals. A sample of each fraction was set aside for analysis.

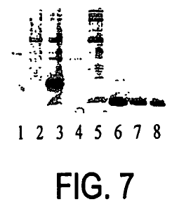

The material was analyzed by SDS-PAGE using a 12% Tris/Glycine gel at a constant 200 volts for 45 minutes. Results are shown in FIG. 7. The pure fractions (lanes 7-8) were loaded into a 3000 MWCO Slidalyzer™ dialysis cassette. The protein was dialyzed at 4° C. into 50 mM sodium phosphate pH 7.0. Two buffer changes ensured complete removal of the Tris buffer. The final protein concentration was determined using UV absorbance at 280 nm; comparing it to the extinction coefficient for MUP (0.503 at 1 mg/mL). The final concentration of pure Murine Urinary Protein was 48 mg/mL in 1.9 mL. Total yield was 90 mg. The final MUP sample was stored at 4° C. prior to NMR analysis.

### Example 3

### NMR Analysis of Murine Urinary Protein (MUP) Containing L-valine-α-D-¹²CD(¹³CHD₂)₂

A 15 mg sample of L-valine-α-D-¹²CD(¹³CHD₂)₂ labeled MUP was dissolved in 650 μL phosphate buffered saline (10 mM potassium phosphate; 200 mM sodium chloride), to which was added 50 μL deuterium oxide. ¹³C Relaxation rates (R₂ and R₁) of ¹³CHD₂ groups were determined using pulse sequences as described in Ishima et al., J. Am. Chem. Soc. 121:11589-11590 (1999). Spectral parameters were as follows: spectral width in the ¹³C dimension, 900 Hz; spectral width in the ¹H dimension, 5200 Hz; number of real data points in the ¹³C dimension, 128; number of real points in the ¹H dimension, 1408; number of transients per t₁ increment, 8; probe temperature, 298 K. Prior to two-dimensional Fourier transformation, free induction decays were apodized with cosine-bell windowing functions according to known methods.

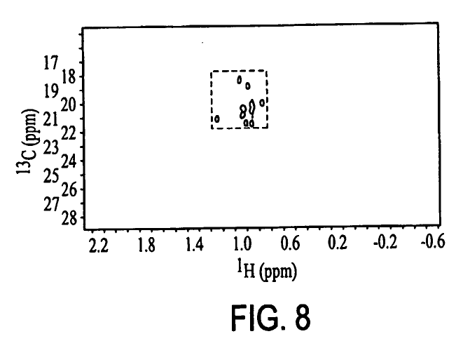

The NMR analysis was then repeated following addition of the small molecule ligand 1 μL of isobutyl pyrazine or 1 μL of isopropyl pyrazine to separate solutions of MUP. The data shown below in Tables I and II and in FIG. 8 were obtained, clearly showing the changes in T1 and T2 values obtained on addition of these small molecule ligands. The relaxation data obtained in the presence of the ligands 2-methoxy-3-isopropylpyrazine and 2-methoxy-3-isobutylpyrazine indicates that valine side-chains do not contribute significantly to the entropy of binding.

TABLE I
Valine (-13CHC2) Labeled Mouse Urinary Protein T1 and T2
Relaxation Times in the Absence and Presence of
Isobutylpyrazine Ligand.

| | | | | |
|---|---|---|---|---|
| | | Bound | | |
| Protein | Error | Protein | Error | Change |

T1 values (milliseconds)

| | | | | | |
|---|---|---|---|---|---|
| 12.HG1 | 1724.922 | 26.602 | 1760.141 | 85.914 | 35.219 |
| 47.HG1 | 1628.719 | 23.207 | 1619.169 | 47.052 | 9.550 |
| 47.HG2 | 990.621 | 25.903 | 1072.690 | 45.334 | −82.069 |
| 53.HG1 | 2233.271 | 38.470 | 2332.247 | 82.853 | −98.976 |
| 53.HG2 | 1364.726 | 21.978 | 1382.566 | 58.719 | 17.840 |
| 59.HG1 | 1282.763 | 21.852 | 1367.075 | 67.324 | −84.312 |
| 59.HG1 | 1532.137 | 36.468 | 1502.825 | 69.414 | 20.312 |
| 70.HG1 | 2081.572 | 27.198 | 1701.953 | 57.518 | 379.619 |
| 70.HG2 | 1163.528 | 15.853 | 1488.130 | 82.016 | −324.602 |
| 82.HG1 | 914.901 | 15.870 | 913.641 | 27.002 | 1.260 |
| 82.HG2 | 669.801 | 11.195 | 703.150 | 26.093 | −33.349 |

T2 values (milliseconds)

| | | | | | |
|---|---|---|---|---|---|
| 12.HG1 | 153.108 | 2.257 | 166.608 | 5.221 | −13.500 |
| 12.HG2 | 183.314 | 2.276 | 200.110 | 6.007 | −16.796 |
| 47.HG1 | 155.232 | 1.994 | 171.287 | 5.421 | −16.055 |
| 47.HG2 | 150.754 | 3.469 | 162.950 | 5.232 | −12.196 |
| 53.HG1 | 163.699 | 2.187 | 174.600 | 4.773 | −10.901 |
| 53.HG2 | 150.283 | 2.148 | 163.379 | 4.812 | −13.096 |
| 59.HG1 | 178.648 | 2.787 | 200.992 | 7.122 | −22.344 |
| 59.HG1 | 147.716 | 3.338 | 150.756 | 6.220 | −3.040 |
| 70.HG1 | 121.202 | 1.399 | 186.968 | 5.222 | −65.766 |
| 70.HG2 | 162.033 | 2.083 | 128.164 | 5.129 | 33.869 |
| 82.HG1 | 224.383 | 3.731 | 216.738 | 5.185 | 7.645 |
| 82.HG2 | 223.453 | 3.198 | 201.054 | 5.870 | 22.399 |

TABLE II
Valine (-¹³(HD₂) Labeled Mouse Urinary Protein
T1 and T2 Relaxation Times in the Absence and
Presence of Isopropylpyrazine Ligand

| | | | | |
|---|---|---|---|---|
| | | Bound | | |
| Protein | Error | Protein | Error | Change |

T1 values (milliseconds)

| | | | | | |
|---|---|---|---|---|---|
| 12.HG1 | 1724.922 | 26.602 | 1733.309 | 39.761 | −8.387 |
| 12.HG2 | 1468.411 | 20.239 | 1509.370 | 24.959 | −40.959 |
| 47.HG1 | 1628.719 | 23.207 | 1626.298 | 23.067 | 2.421 |
| 47.HG2 | 990.621 | 25.903 | 1067.605 | 23.922 | −76.984 |
| 53.HG1 | 2233.271 | 38.470 | 2283.543 | 38.045 | −50.272 |
| 53.HG2 | 1364.726 | 21.978 | 1355.764 | 28.603 | 8.962 |
| 59.HG1 | 1282.763 | 21.852 | 1374.428 | 32.978 | −91.665 |
| 59.HG1 | 1523.137 | 36.468 | 1572.793 | 35.240 | −49.656 |
| 70.HG1 | 2081.572 | 27.198 | 1872.254 | 27.668 | 209.318 |
| 70.HG2 | 1163.528 | 15.853 | 1077.097 | 22.081 | 86.431 |
| 82.HG1 | 914.901 | 15.870 | 925.102 | 13.185 | −10.201 |
| 82.HG2 | 669.801 | 11.195 | 668.810 | 12.161 | 0.991 |

T2 values (milliseconds)

| | | | | | |
|---|---|---|---|---|---|
| 12.HG1 | 153.108 | 2.257 | 161.437 | 3.401 | −8.329 |
| 12.HG2 | 183.314 | 2.276 | 192.512 | 3.174 | −9.198 |
| 47.HG1 | 155.232 | 1.994 | 168.574 | 2.716 | −13.342 |
| 47.HG2 | 150.754 | 3.469 | 155.315 | 3.433 | −4.561 |
| 53.HG1 | 163.699 | 2.187 | 177.873 | 2.505 | −14.174 |
| 53.HG2 | 150.283 | 2.148 | 158.149 | 3.377 | −7.866 |
| 59.HG1 | 178.648 | 2.787 | 195.222 | 4.380 | −16.574 |
| 59.HG1 | 147.716 | 3.338 | 153.897 | 3.555 | −6.181 |
| 70.HG1 | 121.202 | 1.399 | 162.943 | 2.001 | −41.741 |
| 70.HG2 | 162.033 | 2.083 | 150.103 | 2.892 | 11.930 |
| 82.HG1 | 224.383 | 3.731 | 213.526 | 3.037 | 10.857 |
| 82.HG2 | 223.453 | 3.198 | 203.529 | 3.774 | 19.924 |

### Example 4

### NMR Relaxation Measurements of [¹³C^{γ1},^{γ2}HD₂]-U-²H Valine

Samples of [¹³C^{γ1},^{γ2}HD₂]-U-²H valine enriched MUP both alone and complexed with 2-methoxy-3-isopropylpyrazine and 2-methoxy-3-isobutylpyrazine were prepared from a single sample at pH 7.0 and a protein concentration of 1 mM. A further sample of methyl-¹³C, ²H enriched MUP was prepared at pH 7.0 and a protein concentration of 0.5 mM. Longitudinal (R₁) and transverse (R₂) ¹³C relaxation rates were determined essentially as described by Ishima et al., J. Am. Chem. Soc. 121:11589-11590, 1999. All spectra were recorded at a proton frequency of 600 MHz and a probe temperature of 30° C. R₁ rates were determined with relaxation delay times of 16, 64, 128, 240, 400, 720, 1120, 1600, 2240 and 2880 milliseconds. R₂ rates were determined with relaxation delay times of 16, 32, 48, 64, 96, 160, 192, 240 and 288 milliseconds, and an effective field strength of 2 kHz. Relaxation data were fit to a single exponential decay function I=I₀exp(−tR), where I₀ is the initial resonance intensity, t is the relaxation delay time and R is the relaxation rate (R₁ or R₂). Relaxation data were fitted to the Lipari-Szabo model-free spectral density of the form: J(ω)=S²τₘ/(1+ω²τ²ₘ)+(1−S²)τᵢ/(1+ω²τᵢ²), where τᵢ⁻¹=τ_{M}⁻¹+τₑ⁻¹, τ_{M} is the overall molecular tumbling correlation time and τₑ is the effective internal correlation time. See Lipari and Szabo, J. Am. Chem. Soc. 104:4546-4559, 1982. This equation is valid for fast internal motions, τₑ<<τ_{M}, under which conditions the order parameter for methyl CH dipolar relaxation is given by S²=Sₐₓᵢₛ²[P₂(cos θ_{H}]². Sₐₓᵢₛ² is the order parameter of the methyl rotation axis and θ_{H} is the angle made by the axis and the CH bond vector. A global rotational correlation time of 8:57 nanoseconds was used for these calculations, according to the previously reported value. Zidek et al., Nature Str. Biol. 6:1118-1121, 1999. Although the valine residues were perdeuterated at non-methyl positions, the protein was otherwise protonated, and dipolar relaxation due to these “external” protons is not negligible for ¹³C relaxation. Thus, by analogy with the work of Ishima et al. J. Am. Chem. Soc. 123:6164-6171, 2001, the contribution of these external protons to ¹³C R₂ values was estimated at 25% from the X-ray coordinates of MUP. Consequently, measured ¹³C R² rates were multiplied by 0.75 to account for these external dipolar relaxation processes.

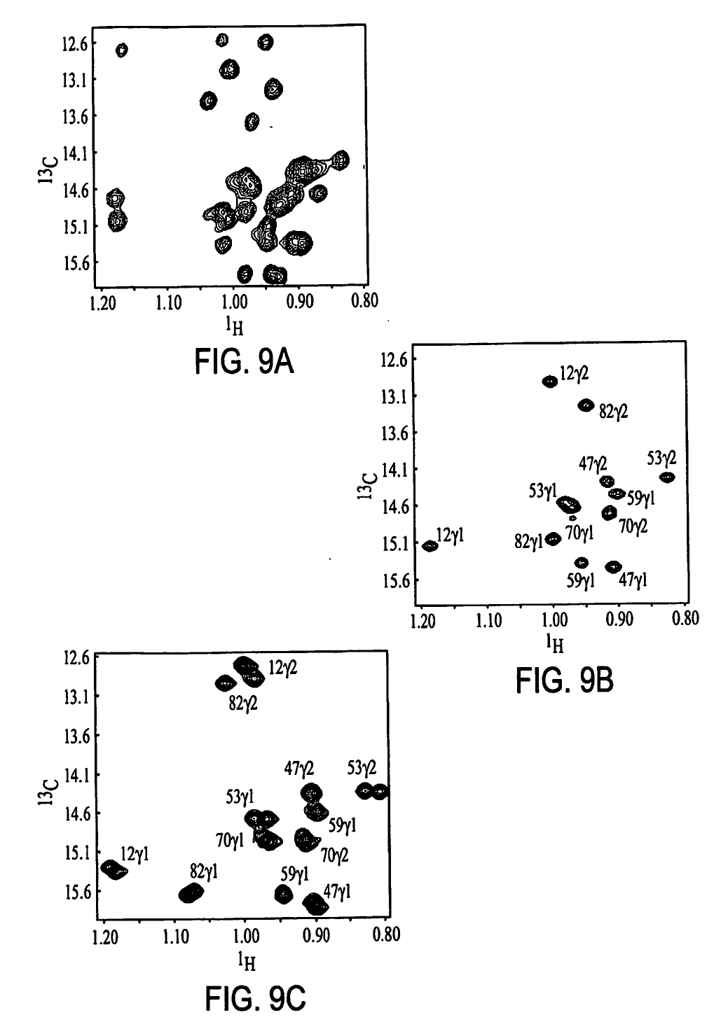

FIG. 9A shows a region from the ¹³C—¹H HSQC spectrum of methyl ¹³C, ²H-enriched mouse major urinary protein, containing valine Cγ1-Hγ1 and Cy2-Hγ2 correlations. Significant overlap is present in the spectrum, arising from the combined effects of interference from resonances from ¹³CH₃ isotopomers, together with methyl resonances derived from residues other than valine. In contrast, an equivalent spectrum recorded on [¹³C^{γ1},^{γ2}HD₂]-U-²H valine enriched MUP (FIG. 9B) is essentially free from resonance overlap, and all twelve valine methyl groups can be observed and assigned. See Abbate et al., J. Biomol. NMR 15:187-188, 1999.

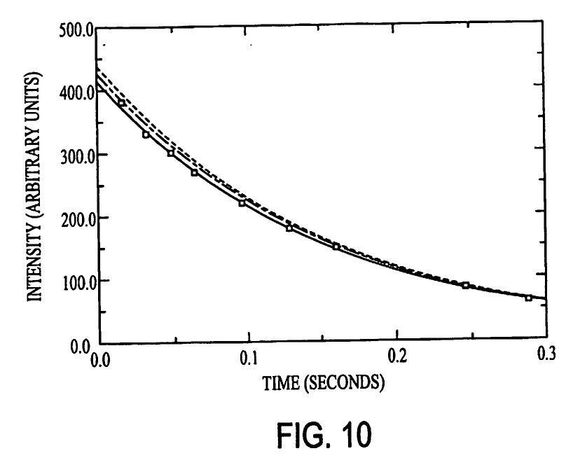

MUP binds to the small hydrophobic ligands, 2-methoxy-3-isopropylpyrazine and 2-methoxy-3-isobutylpyrazine, which bind to MUP with Kd's of 560 nM and 80 nM, respectively. FIG. 9C shows ¹³C—¹H HSQC spectra of complexes of [¹³C^{γ1},^{γ2}HD₂]-U-²H valine-enriched MUP with these ligands. Significant shift perturbations were observed for the correlations from Val 82 γ1 and γ2. This was anticipated since Val 82 is located within the binding pocket of MUP. Timm et al, Prot. Sci. 10:997-1004, 2001. In both complexes all correlations were well resolved, in contrast to complexes with methyl-¹³C, ²H enriched MUP (data not shown). Under these circumstances, measurement of ¹³C longitudinal and transverse relaxation rates (R₁ and R₂) can be undertaken in each sample at high sensitivity and with minimal interference due to resonance overlap. Relaxation curves were created. Typical results are shown in FIG. 10.

TABLE III
S² Values for Valine Methyl Groups Derived from 600 MHz
¹³C T₁ and T₂ Measurements on Major Urinary Protein Selectively
Enriched with [¹³C^{γ1}, ^{γ2}HD2]-U-²H Valine.

| | | Ligand | |
|---|---|---|---|
| | | 2-methoxy-3- | 2-methoxy- |
| | | isopropyl | 3-isobutyl- |
| | None | pyrazine | pyrazine |
| Residue | S²ₐₓᵢₛ* | S²ₐₓᵢₛ | S²ₐₓᵢₛ |
| Val-12 C^{γ1} | 0.88 | 0.90 | 0.86 |
| Val-12 C^{γ2} | 0.77 | 0.69 | 0.66 |
| Val-47 C^{γ1} | 0.91 | 0.84 | 0.86 |
| Val-47 C^{γ2} | 0.86 | 0.90 | 0.88 |
| Val-53 C^{γ1} | 0.74 | 0.80 | 0.78 |
| Val-53 C^{γ2} | 0.90 | 0.85 | 0.86 |
| Val-59 C^{γ1} | 0.73 | 0.68 | 0.63 |
| Val-59 C^{γ2} | 0.98 | 0.86 | 0.90 |
| Val-70 C^{γ1} | 0.78 | 0.85 | 0.81 |
| Val-70 C^{γ2} | 0.77 | 0.77 | [1.03] |
| Val-82 C^{γ1} | 0.53 | 0.50 | 0.53 |
| Val-82 C^{γ2} | 0.46 | 0.48 | 0.53 |

*The average error in the reported S²axis values 0.045 +/− 0.009. The bracketed value is anomalous due to strong coupling between Val-70 C^{γ2} and C^{γ1}.

Values of S²ₐₓᵢₛ were derived from R₁ and R₂ data for each methyl group using the model-free spectral density function given above, and are listed in Table III. There were only minor changes Sₐₓᵢₛ² for any of the six valine residues in MUP upon binding of either 2-methoxy-3-isopropylpyrazine or 2-methoxy-3-isobutylpyrazine, most of which are within experimental error. An exception is Sₐₓᵢₛ² for Val-70 C^{γ}2, which appears to increase dramatically on binding 2-methoxy-3-isobutylpyrazine. However, this is an anomalous result—Val-70 C^{γ}2, which appears to increase dramatically on binding 2-methoxy-3-isobutylpyrazine. However, this is an anomalous result—Val-70 C^{γ}1 and C^{γ}2 possess similar shifts in both complexes, and differ by ˜3 Hz in the 2-methoxy-3-isobutylpyrazine complex, under which conditions C^{γ}1 and C^{γ}2 will be strongly coupled via the two bond homonuclear scalar coupling. This results in a poor fit of R2 relaxation data for Val-70 C^{γ}2 (χ²=49.7).

Measured S²ₐₓᵢₛ values for the two methyl groups of certain valine residue are not identical within experimental error. At first sight this is inconsistent with the requirement for their mobilities to be essentially be the same, since they form part of the same isopropyl group. However, this is not necessarily reflected in equivalent S²ₐₓᵢₛ values. Order parameters of methyl groups from the same isopropyl group may differ if the effective averaging axis for this group makes different angles with the methyl threefold axes.

The constant values of Sₐₓᵢₛ² on complex formation for the methyl groups of Val-82 is unexpected since this residue is located within the binding pocket and is proximal to the bound ligands. Intuitively, the relevant Sₐₓᵢₛ² values might be expected to increase due to decreased mobility within the binding pocket in the complexes. Since each ligand is protonated, additional relaxation pathways offered by ligand protons are offset by an equal opposite effective contribution due to increased mobility of Val-82 side-chain atoms. In contrast, remaining valine methyl groups are distal to the binding pocket, and ligand protons will make a negligible contribution to relaxation in these cases, since no significant structural changes in MUP are observed in the crystal structures of these complexes (data not shown).

Previous studies using ²H relaxation methods on calcium-saturated calmodulin in complex with a peptide model of the calmodulin-binding domain have highlighted significant changes in Sₐₓᵢₛ² values that cannot be predicted in a rational manner. Changes to Sₐₓᵢₛ² values were found at locations both proximal and distal from the Ca²⁺ binding site. The present observations suggest that this phenomenon may not be general. Indeed, the dominant entropic contribution to binding from protein dynamics may derive from the backbone, as has been suggested for the complex between MUP and a small-molecule ligand unrelated to the pyrazine-derivatives described here. Zidek et al., Nature Str. Biol. 6:1118-1121, 1999. Computations of Sₐₓᵢₛ² values for each of the valine methyl groups both in the free protein and in complexes with 2-methoxy-3-isopropylpyrazine and 2-methoxy-3-isobutylpyrazine indicate that valine side-chains do not contribute significantly to the entropy of binding in this case. Since the methods according to the invention are highly sensitive, lower (10-20%) levels of ¹³C-enrichment may be used. Levels of about 5-30% are appropriate, or levels of about 10-20% and most preferably about 10%.

1. An amino acid wherein at least one bond vector in the side chain of said amino acid consists of two NMR-active nuclei bonded together and wherein essentially all other nuclei are NMR inactive.

2. An amino acid wherein at least one bond vector in the side chain of said amino acid consists of two NMR-active nuclei bonded together and wherein essentially all other bond vectors are NMR inactive.

3. The amino acid of claim 1 or claim 2 wherein said two NMR-active nuclei are ¹³C and ¹H, wherein the remainder of the carbon atoms in said amino acid are essentially ¹²C, wherein the nitrogen atoms in said amino acid are essentially ¹⁴N and wherein the remainder of the hydrogen atoms in said amino acid are essentially ²H.

4. The amino acid of claim 1 or claim 2 wherein said two NMR-active nuclei are ¹³C and ¹H, wherein the remainder of the carbon atoms in said amino acid are essentially ¹²C, wherein the nitrogen atoms in said amino acid are essentially ¹⁴N and wherein the remainder of the hydrogen atoms in said amino acid are natural abundance.

5. The amino acid of claim 1 or claim 2 wherein said two NMR-active nuclei are ¹⁵N and ¹H, wherein the remainder of the nitrogen atoms in said amino acid are essentially ¹⁴N, wherein the carbon atoms in said amino acid are essentially ¹²C and wherein the remainder of the hydrogen atoms in said amino acid are essentially ²H.

6. The amino acid of claim 1 or claim 2 wherein said two NMR-active nuclei are ¹⁵N and ¹H, wherein the remainder of the nitrogen atoms in said amino acid are essentially ¹⁴N, wherein the remainder of the carbon atoms in said amino acid are essentially ¹²C and wherein the remainder of the hydrogen atoms in said amino acid are natural abundance.

7. A culture medium comprising an amino acid of claim 1 or claim 2.

8. A protein comprising at least one amino acid of claim 1 or claim 2.

9. A method for analyzing the dynamics of a bond vector of a protein comprising producing said protein in a form which comprises an amino acid of claim 1 or claim 2 and subjecting said protein to NMR spectroscopy.

10. A method of determining the entropic contribution of a bond vector of a protein bound to a ligand comprising producing said protein in a form which comprises an amino acid of claim 1 or claim 2 and subjecting said protein to NMR spectroscopy in the presence and the absence of said ligand.

11. A method of preparing an isotopically substituted protein comprising culturing cells that express said protein in a medium containing at least one amino acid according to claim 1 or claim 2 and recovering said protein from the cell culture.
